(19) United States
(12) Patent Application Publication
(10) Pub. No.: US 2007/0243149 A1
(43) Pub. Date: Oct. 18, 2007

(54) Nitrocellulose-based binders for aqueous nail varnishes

(76) Inventors: Steffen Hofacker, Odenthal (DE); Thorsten Rische, Unna (DE); Rainer Trinks, Dormagen (DE); Sebastian Dorr, Dusseldorf (DE); Werner Lange, Visselhovede (DE)

Correspondence Address: BAYER MATERIAL SCIENCE LLC, 100 BAYER ROAD, PITTSBURGH, PA, 15205, US

(21) Appl. No.: 11/732,576

(22) Filed: Apr. 4, 2007

(30) Foreign Application Priority Data
Apr. 7, 2006 (DE) 102006016452.0

(51) Int. Cl. A61K 8/73; C08L 75/06

(52) U.S. Cl. 424/61; 424/70.13; 525/440.06

(57) ABSTRACT

The present invention relates to a new aqueous binder system for nail varnishes, based on nitrocellulose-containing polyurethanepolyurea dispersions.

### CROSS-REFERENCE TO RELATED APPLICATIONS

The present application claims the right of priority under 35 U.S.C. § 119 (a)-(d) of German Patent Application Number 10 2006 016 452.0, filed Apr. 8, 2006.

### BACKGROUND OF THE INVENTION

The present invention relates to a new aqueous binder system for nail varnishes, based on dispersions comprising nitrocellulose-polyurethanepolyurea particles.

Current nail varnishes are produced almost exclusively on the basis of solvent-borne, physically drying binders. Nitrocellulose, in particular, is used predominantly as the major ingredient in the solvent-borne binder.

In view of the emerging debate on the reduction of volatile organic solvents in the cosmetics sector, there is great interest in reducing the level of, if not eliminating, solvent fractions in conventional nail varnishes.

Nitrocellulose itself is virtually insoluble in water. It is only by modifying the polymer framework, such as by introducing hydrophilic side groups, for example, that solubility in water can be produced. Changing the polymer framework, however, has an adverse effect on the properties of nitrocellulose that are desirable in the nail varnish sector, such as high gloss.

It is for this reason that attempts have been made to switch to different polymer systems which exhibit solubility in water as well as the other properties required, such as film formation, mechanical properties, etc.

Thus EP-A 0 391 322 describes aqueous nail varnishes based on aqueous polyurethanes and/or polyurethane-acrylate copolymers as binders. WO 2003/039445, furthermore, teaches the use of aqueous polyurethane dispersions for producing nail varnishes free from or low in organic solvent. U.S. Pat. No. 6,391,964 also describes the use of water-based acrylate polymer emulsions in combination with water-based polyurethane resins for producing aqueous nail varnishes. In addition, for example, U.S. Pat. No. 5,955,063 describes aqueous acrylate binders for producing water-based nail varnishes.

A great disadvantage associated with these aqueous binders, however, is that important properties such as gloss, hardness and drying time do not meet the requirements of practice.

Furthermore, U.S. Pat. No. 5,637,292 describes the use of aqueous acrylate polymers having a fraction of acrylate monomers which are brought to reaction by means of UV light after formulation of the nail varnish and which consequently exhibit very rapid drying/curing. A disadvantage with these systems, however, is the presence of acrylate monomers, which must be classed as objectionable from the standpoint of hygiene. Moreover, the effect of UV light may cause tissue damage and should therefore be avoided.

WO 1999/055290 describes, furthermore, the use of film-forming polyurethane polymers in combination with nitrocellulose, albeit using organic solvents and/or plasticizers. Aqueous systems, in contrast, are not described.

It was an object of the present invention, then, to provide new aqueous binders for producing nail varnishes that have organic solvent contents, at maximum, of less than 5% by weight and which do not have disadvantages of aqueous systems of the prior art.

It has been found that the object stated can be achieved through the use of specific dispersions comprising nitrocellulose-polyurethane-polyurea particles.

### SUMMARY OF THE INVENTION

The invention accordingly provides aqueous nail varnishes comprising polyurethane-nitrocellulose particles in the form of an aqueous dispersion (I) having an average particle size of 20 to 700 nm measured by means of laser correlation spectroscopy (Zetasizer 1000, Malvern Instruments, Malvern, UK).

The invention further provides a method of producing aqueous nail varnishes comprising

A) preparing one or more isocyanate-functional prepolymers from A1) one or more organic polyisocyanates
A2) one or more polymeric polyols having number-average molecular weights of 400 to 8000 g/mol and OH functionalities of 1.5 to 6,
A3) one or more hydroxy-functional compounds having molecular weights of 62 to 399 g/mol and
A4) one or more isocyanate-reactive, anionic or potentially anionic and optionally nonionic hydrophilicizing agents,

B) reacting some or all of the free NCO groups, before, during or after the addition of an organic solvent, with
B1) one or more amino-functional compounds having molecular weights of 32 to 399 g/mol and/or
B2) one or more amino-functional, anionic or potentially anionic hydrophilicizing agents, with chain extension,

C) dispersing the prepolymers, before during or after step B), in water, any potentially ionic groups present being converted into the ionic form by full or partial reaction with a neutralizing agent,

D) adding nitrocellulose in the form of a solution in an organic solvent or solvent mixture after step A) but before step C), and

E) distilling the dispersion to remove the organic solvent present.

### DETAILED DESCRIPTION OF THE PREFERRED EMBODIMENTS

The aqueous nail varnishes of the present invention, based on the overall formulation, contain less than 5%, preferably <2%, more preferably <1%, by weight of organic solvents and/or plasticizers.

By plasticizers are meant compounds such as phthalates, castor oil, acetyl tributyl citrate or alkylated phosphates.

The aqueous dispersions (I) are obtainable by

A) preparing one or more isocyanate-functional prepolymers from
A1) one or more organic polyisocyanates
A2) one or more polymeric polyols having number-average molecular weights of 400 to 8000 g/mol, preferably 400 to 6000 g/mol and more preferably of 600 to 3000 g/mol, and OH functionalities of 1.5 to 6, preferably 1.8 to 3, more preferably of 1.9 to 2.1,
A3) one or more hydroxy-functional compounds having molecular weights of 62 to 399 g/mol and
A4) one or more isocyanate-reactive, anionic or potentially anionic and optionally nonionic hydrophilicizing agents,

B) reacting some or all of the free NCO groups, before, during or after the addition of an organic solvent, with
B1) one or more amino-functional compounds having molecular weights of 32 to 399 g/mol and/or
B2) one or more amino-functional, anionic or potentially anionic hydrophilicizing agents, with chain extension,

C) dispersing the prepolymers, before during or after step B), in water, any potentially ionic groups present being converted into the ionic form by full or partial reaction with a neutralizing agent

D) adding nitrocellulose in the form of a solution in an organic solvent or solvent mixture after step A) but before step C), and

E) distilling the dispersion to remove the organic solvent present.

Preferred organic solvents for preparing the dispersions essential to the invention are aliphatic ketones, more preferably acetone or 2-butanone.

Suitable polyisocyanates of component A1) are the aromatic, araliphatic, aliphatic or cycloaliphatic polyisocyanates with an NCO functionality of ≧2 that are known per se to the skilled person.

Examples of suitable such polyisocyanates are 1,4-butylene diisocyanate, 1,6-hexamethylene diisocyanate (HDI), isophorone diisocyanate (IPDI), 2,2,4-and/or 2,4,4-trimethylhexamethylene diisocyanate, the isomeric bis(4,4′-isocyanato-cyclohexyl)methanes or their mixtures with any desired isomer content, 1,4-cyclohexylene diisocyanate, 1,4-phenylene diisocyanate, 2,4- and/or 2,6-tolylene diisocyanate, 1,5-naphthylene diisocyanate, 2,2′- and/or 2,4′- and/or 4,4′-diphenyl-methane diisocyanate, 1,3- and/or 1,4-bis(2-isocyanatoprop-2-yl)benzene (TMXDI), 1,3-bis(isocyanatomethyl)benzene (XDI), (S)-alkyl 2,6-diiso-cyanatohexanoates, (L)-alkyl 2,6-diisocyanatohexanoates, having branched, cyclic or acyclic alkyl groups with up to 8 carbon atoms.

Besides the abovementioned polyisocyanates it is also possible to use as well, proportionally, modified diisocyanates having a uretdione, isocyanurate, urethane, allophanate, biuret, iminooxadiazinedione and/or oxadiazinetrione structure, and also non-modified polyisocyanate having more than 2 NCO groups per molecule e.g. 4-isocyanatomethyl-1,8-octane diisocyanate (nonane triisocyanate) or triphenylmethane 4,4′,4″-triisocyanate.

The polyisocyanates or polyisocyanate mixtures in question are preferably those of the abovementioned kind having exclusively aliphatically and/or cycloaliphatically attached isocyanate groups and having an average NCO functionality, of the mixture, of 2 to 4, preferably 2 to 2.6 and more preferably 2 to 2.4.

Particular preference is given to using in A1) hexamethylene diisocyanate, isophorone diisocyanate, the isomeric bis(4,4′-isocyanatocyclohexyl)methanes, and mixtures thereof.

In A2) polymeric polyols are used which have a number-average molecular weight. Mₙ of 400 to 8000 g/mol, preferably of 400 to 6000 g/mol and more preferably of 600 to 3000 g/mol. These polyols preferably have an OH functionality of 1.5 to 6, more preferably of 1.8 to 3, very preferably of 1.9 to 2.1.

Polymeric polyols of this kind are polyurethane coating technology's conventional polyester polyols, polyacrylate polyols, polyurethane polyols, polycarbonate polyols, polyether polyols, polyesterpolyacrylate polyols, polyurethanepolyacrylate polyols, polyurethanepolyester polyols, polyurethanepolyether polyols, polyurethanepolycarbonate polyols, polyesterpolycarbonate polyols and phenol/formaldehyde resins. They can be used in A2) individually or in any desired mixtures with one another.

Polyester polyols of this kind are the conventional polycondensates of di- and also optionally tri- and tetraols and di- and also optionally tri- and tetracarboxylic acids or hydroxycarboxylic acids or lactones. In place of the free polycarboxylic acids it is also possible to use the corresponding polycarboxylic anhydrides or corresponding polycarboxylic esters of lower alcohols for preparing the polyesters.

Examples of suitable diols are ethylene glycol, butylene glycol, diethylene glycol, triethylene glycol, polyalkylene glycols such as polyethylene glycol, and 1,2-propanediol, 1,3-propanediol, butane-1,3-diol, butane-1,4-diol, hexane-1,6-diol and isomers, neopentyl glycol or neopentyl glycol hydroxypivalate, preference being given to hexane-1,6-diol and isomers, neopentyl glycol and neopenthyl glycol hydroxypivalate. In addition it is also possible to use polyols such as trimethylolpropane, glycerol, erythritol, pentaerythritol, trimethylolbenzene or trishydroxyethyl isocyanurate.

Dicarboxylic acids which can be used include phthalic acid, isophthalic acid, terephthalic acid, tetrahydrophthalic acid, hexahydrophthalic acid, cyclohexanedicarboxylic acid, adipic acid, azelaic acid, sebacic acid, glutaric acid, tetrachlorophthalic acid, maleic acid, fumaric acid, itaconic acid, malonic acid, suberic acid, 2-methylsuccinic acid, 3,3-diethylglutaric acid and/or 2,2-dimethyl-succinic acid. The acid source used can also be the corresponding anhydrides.

Where the average functionality of the polyol to be esterified is >2, it is additionally also possible as well to use monocarboxylic acids, such as benzoic acid and hexanecarboxylic acid.

Preferred acids are aliphatic or aromatic acids of the abovementioned kind. Particularly preferred are adipic acid, isophthalic acid and optionally trimellitic acid.

Hydroxycarboxylic acids, which can be used as well as reactants when preparing a polyester polyol having terminal hydroxyl groups, are, for example, hydroxycaproic acid, hydroxybutyric acid, hydroxydecanoic acid, hydroxystearic acid and the like. Suitable lactones are caprolactone, butyrolactone and homologues. Caprolactone is preferred.

It is likewise possible in A2) to use hydroxyl-containing polycarbonates, preferably polycarbonatediols, having number-average molecular weights Mₙ of 400 to 8000 g/mol, preferably 600 to 3000 g/mol. These polycarbonates are obtainable by reacting carbonic acid derivatives, such as diphenyl carbonate, dimethyl carbonate or phosgene, with polyols, preferably diols.

Examples of such diols are ethylene glycol, 1,2- and 1,3-propanediol, 1,3- and 1,4-butanediol, 1,5-pentanediol, 1,6-hexanediol, 1,8-octanediol, neopentyl glycol, 1,4-bishydroxymethylcyclohexane, 2-methyl-1,3-propanediol, 2,2,4-trimethylpentane-1,3-diol, 3-methyl-1,5-pentanediol, dipropylene glycol, polypropylene glycols, dibutylene glycol, polybutylene glycols, bisphenol A, tetrabromobisphenol A and lactone-modified diols of the abovementioned kind.

Preferably the diol component contains 40% to 100% by weight of hexanediol, preference being given to 1,6-hexanediol and/or hexanediol derivatives. Hexanediol derivatives of this kind are based on hexanediol and besides terminal OH groups contain ester groups or ether groups. Derivatives of this kind are obtainable by reacting hexanediol with excess caprolactone or by etherifying hexanediol with itself to give the di- or trihexylene glycol.

Instead of, or in addition to, plain polycarbonate diols, it is also possible to use polyether-polycarbonate diols in A2).

Polycarbonates containing hydroxyl groups are preferably of linear construction, but may also be obtained easily through the incorporation of polyfunctional components, especially low molecular weight polyols. Examples of those suitable for this purpose include glycerol, trimethylolpropane, hexane-1,2,6-triol, butane-1,2,4-triol, trimethylolpropane, trimethylolethane, pentaerythritol, quinitol, mannitol, sorbitol, methylglycoside or 1,3,4,6-dianhydrohexitols.

It is likewise possible in A2) to use polyether polyols. Suitability is possessed, for example, by the polytetramethylene glycol polyethers known per se in polyurethane chemistry, of the kind obtainable by polymerizing tetrahydrofuran by means of cationic ring opening.

Polyether polyols likewise suitable are the conventional adducts of styrene oxide, ethylene oxide, propylene oxide, butylene oxides and/or of epichlorohydrin with difunctional or polyfunctional starter molecules.

Suitable starter molecules which can be used are all compounds known from the prior art, such as, for example, water, butyldiglycol, glycerol, diethylene glycol, trimethylolpropane, propylene glycol, sorbitol, ethylenediamine, triethanolamine, 1,4-butanediol.

Preference is given to using as A2) polyester polyols, polytetramethylene glycol polyethers and/or polycarbonate polyols.

In A3) it is possible to use polyols having molecular weights of 62 to 399 g/mol and having up to 20 carbon atoms. These may be ethylene glycol, diethylene glycol, triethylene glycol, 1,2-propanediol, 1,3-propanediol, 1,4-butanediol, 1,3-butylene glycol, cyclohexanediol, 1,4-cyclohexanedimethanol, 1,6-hexanediol, neopentyl glycol, hydroquinone dihydroxyethyl ether, bisphenol A (2,2′-bis(4-hydroxyphenyl)propane), hydrogenated bisphenol A (2,2′-bis(4-hydroxycyclohexyl)propane, trimethylolpropane, glycerol, pentaerythritol and also any desired mixtures thereof with one another.

Also suitable are esterdiols of the stated molecular weight range such as E-hydroxy-caproic α-hydroxybutyl ester, γ-hydroxybutyric (ω-hydroxyhexyl ester, adipic acid (β-hydroxyethyl) ester or terephthalic acid bis(β-hydroxyethyl) ester.

Furthermore it is possible in A3) to use monofunctional isocyanate-reactive hydroxyl-containing compounds as well. Examples of such monofunctional compounds are ethanol, n-butanol, ethylene glycol monobutyl ether, diethylene glycol monomethyl ether, ethylene glycol monobutyl ether, diethylene glycol monobutyl ether, propylene glycol monomethyl ether, dipropylene glycol monomethyl ether, tripropylene glycol monomethyl ether, dipropylene glycol monopropyl ether, propylene glycol monobutyl ether, dipropylene glycol monobutyl ether, tripropylene glycol monobutyl ether, 2-ethylhexanol, 1-octanol, 1-dodecanol, 1-hexadecanol.

By anionically or potentially anionically hydrophilicizing compounds of component A4) are meant all compounds which have at least one isocyanate-reactive hydroxyl group and also at least one functionality, such as —COOY, —SO₃Y, —PO(OY)₂, (Y for example ═H⁺, NH₄⁺, metal cation), which on interaction with aqueous media enters into a pH-dependent dissociation equilibrium and in this way may carry a negative or neutral charge. Suitable anionically or potentially anionically hydrophilicizing compounds are mono- and dihydroxycarboxylic acids, mono- and dihydroxysulphonic acids, and also mono- and dihydroxyphosphonic acids and their salts. Examples of such anionic or potentially anionic hydrophilicizing agents are diemethylolpropionic acid, dimethylolbutyric acid, hydroxypivalic acid, malic acid, citric acid, glycolic acid, lactic acid, and the adduct of sodium bisulphite with but-2-ene-1,4-diol, polyethersulphonate and the propoxylated adduct of 2-butenediol and NaHSO₃, as described in DE-A 2 446 440, page 5-9, formula I-III. Preferred anionic or potentially anionic hydrophilicizing agents of component A4) are those of the abovementioned kind which possess carboxyl or carboxylate and/or sulphonate groups.

Particularly preferred anionic or potentially anionic hydrophilicizing agents are those which contain carboxyl groups and/or sulphonate groups as ionic or potentially ionic groups, such as the salts of dimethylolpropionic acid or dimethylolbutyric acid.

Suitable nonionically hydrophilicizing compounds of component A4) are, for example, polyoxyalkylene ethers which contain at least one hydroxyl or amino group.

Examples are the monohydroxy-functional polyalkylene oxide polyether alcohols that contain on average 5 to 70, preferably 7 to 55, ethylene oxide units per molecule, of the kind obtainable in conventional manner by alkoxylating suitable starter molecules (e.g. in Ullmanns Encyclopädie der technischen Chemie, 4th edition, Volume 19, Verlag Chemie, Weinheim pp. 31-38).

These are either plain polyethylene oxide ethers or mixed polyalkylene oxide ethers, containing at least 30 mol %, preferably at least 40 mol %, based on all of the alkylene oxide units present, of ethylene oxide units.

Particularly preferred nonionic compounds are monofunctional mixed polyalkylene oxide polyethers which contain 40 to 100 mol % ethylene oxide units and 0 to 60 mol % propylene oxide units.

Suitable starter molecules for such nonionic hydrophilicizing agents are saturated monoalcohols such as methanol, ethanol, n-propanol, isopropanol, n-butanol, isobutanol, sec-butanol, the isomeric pentanols, hexanols, octanols and nonanols, n-decanol, n-dodecanol, n-tetradecanol, n-hexadecanol, n-octadecanol, cyclohexanol, the isomeric methylcyclohexanols or hydroxymethylcyclohexane, 3-ethyl-3-hydroxymethyloxetane or tetrahydrofurfuryl alcohol, diethylene glycol monoalkyl ethers, such as diethylene glycol monobutyl ether, unsaturated alcohols such as allyl alcohol, 1,1-dimethylallyl alcohol or oleyl alcohol, aromatic alcohols such as phenol, the isomeric cresols or methoxyphenols, araliphatic alcohols such as benzyl alcohol, anisyl alcohol or cinnamyl alcohol, secondary monoamines such as dimethylamine, diethylamine, dipropylamine, diisopropylamine, dibutylamine, bis(2-ethylhexyl)amine, N-methyl- and N-ethylcyclohexylamine or dicyclohexylamine and also heterocyclic secondary amines such as morpholine, pyrrolidine, piperidine or 1H-pyrazole. Preferred starter molecules are saturated monoalcohols of the abovementioned kind. Particular preference is given to using diethylene glycol monobutyl ether or n-butanol as starter molecules.

Alkylene oxides suitable for the alkoxylation reaction are, in particular, ethylene oxide and propylene oxide, which can be used in any order or else in a mixture for the alkoxylation reaction.

As component B1) it is possible to use diamines or polyamines such as 1,2-ethylenediamine, 1,2- and 1,3-diaminopropane, 1,4-diaminobutane, 1,6-diaminohexane, isophoronediamine, isomer mixture of 2,2,4- and 2,4,4-trimethyl-hexamethylenediamine, 2-methylpentamethylenediamine, diethylenetriamine, 1,3- and 1,4-xylylenediamine, α,α,α′,α′-tetramethyl-1,3- and -1,4-xylylenediamine and 4,4-diaminodicyclohexylmethane and/or dimethylethylenediamine. Likewise possible is the use of hydrazine or hydrazides such as adipic dihydrazide.

As component B1) it is also possible, furthermore, to use compounds which as well as a primary amino group also contain secondary amino groups or as well as an amino group (primary or secondary) also contain OH groups. Examples thereof are primary/secondary amines, such as diethanolamine, 3-amino-1-methylaminopropane, 3-amino-1-ethylaminopropane, 3-amino-1-cyclohexylaminopropane, 3-amino-1-methylaminobutane, alkanolamines such as N-aminoethylethanolamine, ethanolamine, 3-aminopropanol, neopentanolamine.

As component B1) it is also possible, furthermore, to use monofunctional isocyanate-reactive amine compounds, such as methylamine, ethylamine, propylamine, butylamine, octylamine, laurylamine, stearylamine, isononyloxypropylamine, dimethylamine, diethylamine, dipropylamine, dibutylamine, N-methylaminopropylamine, diethyl(methyl)aminopropylamine, morpholine, piperidine, and/or suitable substituted derivatives thereof, amide amines formed from diprimary amines and monocarboxylic acids, monoketimes of diprimary amines, primary/tertiary amines, such as N,N-dimethylaminopropylamine.

By anionically or potentially anionically hydrophilicizing compounds of component B2) are meant all compounds which have at least one isocyanate-reactive amino group and also at least one functionality, such as —COOY, —SO₃Y, —PO(OY)₂, (Y for example ═H⁺, NH₄⁺, metal cation), which on interaction with aqueous media enters into a pH-dependent dissociation equilibrium and in this way may carry a negative or neutral charge.

Suitable anionically or potentially anionically hydrophilicizing compounds are mono- and diaminocarboxylic acids, mono- and diaminosulphonic acids and mono- and diaminophosphonic acids and their salts. Examples of such anionic or potentially anionic hydrophilicizing agents are N-(2-aminoethyl)-β-alanine, 2-(2-aminoethylamino)ethanesulphonic acid, ethylenediamine-propyl- or -butylsulphonic acid, 1,2- or 1,3-propylenediamine-β-ethylsulphonic acid, glycine, alanine, taurine, lysine, 3,5-diaminobenzoic acid and the adduct of IPDI and acrylic acid (EP-A 0 916 647, Example 1), Additionally it is possible to use cyclohexylaminopropanesulphonic acid (CAPS) from WO-A 01/88006 as an anionic or potentially anionic hydrophilicizing agent.

Preferred anionic or potentially anionic hydrophilicizing agents of component B2) are those of the abovementioned kind which possess carboxyl or carboxylate and/or sulphonate groups.

Particularly preferred anionic or potentially anionic hydrophilicizing agents B2) are those which contain carboxylate groups and/or sulphonate groups as ionic or potentially ionic groups, such as the salts of N-(2-aminoethyl)-β-alanine, of 2-(2-0.1% to 25% by weight of components A4) and B2), there being used 0.1% to 5% by weight of anionic or potentially anionic hydrophilicizing agents A4) and B2), based on the total amounts of components A1) to A4) and B1) to B2).

In one particularly preferred embodiment components A1) to A4) and B1) to B2) are used in the following amounts, the individual amounts adding up in each case to 100% by weight:

5% to 35% by weight of component A1),

60% to 90% by weight of component A2),

0.5% to 15% by weight of components A3) and B1), and

0.1% to 15% by weight of components A4) and B2), there being used 0.2% to 4% by weight of anionic or potentially anionic hydrophilicizing agents A4) and B2), based on the total amounts of components A1) to A4) and B1) to B2).

In one very particularly preferred embodiment components A1) to A4) and B1) to B2) are used in the following amounts, the individual amounts adding up in each case to 100% by weight:

10% to 30% by weight of component A1),

65% to 85% by weight of component A2),

0.5% to 14% by weight of components A3) and B1), and

0.1% to 13.5% by weight of components A4) and B2), there being used 0.5% to 3.0% by weight of anionic or potentially anionic hydrophilicizing agents based on the total amounts of components A1) to A4).

In the neutralizing step C) for the full or partial conversion of potentially anionic groups into anionic groups use is made of bases such as tertiary amines, e.g. trialkylamines having 1 to 12, preferably 1 to 6, carbon atoms in each alkyl radical, or of alkali metal bases such as the corresponding hydroxides.

Examples thereof are trimethylamine, triethylamine, methyldiethylamine, tripropylamine, N-methylmorpholine, methyldiisopropylamine, ethyldiiso-propylamine and diisopropylethylamine. The alkyl radicals may for example also carry hydroxyl groups, as in the case of the dialkylmonoalkanolamines, alkyldialkanolamines and trialkanolamines. Neutralizing agents which can be used optionally also include inorganic bases, such as aqueous ammonia solution or sodium or potassium hydroxide.

Preference is given to ammonia, triethylamine, triethanolamine, dimethylethanolamine or diisopropylethylamine, and sodium hydroxide.

The molar amount of the bases is generally 50 and 125 mol %, preferably between 70 and 100 mol % of the molar amount of the acid groups to be neutralized. Neutralization may also take place simultaneously with dispersing, with the dispersing water already containing the neutralizing agent.

Dispersing in water in accordance with step C) takes place preferably subsequent to the chain extension.

For dispersing in water, either the dissolved and chain-extended polyurethane polymer is introduced into the dispersing water, where appropriate with strong shearing, such as vigorous stirring, for example, or, conversely, the dispersing water is stirred into the chain-extended polyurethane polymer solutions. With preference the water is added to the dissolved, chain-extended polyurethane polymer.

Suitable nitrocellulose for use in step D) is water-insoluble nitrocellulose at any nitrogen content or viscosity level. Of preferential suitability are nitrocelluloses which feature, for example, the typical collodion grades (on the term “Collodion” cf. Römpp's Chemielexikon, Thieme Verlag, Stuttgart), i.e. cellulose-nitric esters, having a nitrogen content of 10% to 12.8% by weight, preferably a nitrogen fraction of 10.7% to 12.3% by weight, based on nitrocellulose dry matter.

Particular preference is given to cellulose-nitric esters having a nitrogen content of 10.7% to 12.6%, very preferably of 10.7% to 12.3% by weight. Examples of cellulose-nitric esters of this kind are the Walsroder® nitrocellulose A products (Wolff Cellulosics GmbH & Co. KG, Bomlitz DE) having a nitrogen content of 10.7% to 11.3% by weight, or Walsroder® nitrocellulose AM products (Wolff Cellulosics GmbH & Co. KG, Bomlitz, DE), which have a nitrogen content of 11.3% to 11.8% by weight, or Walsroder® nitrocellulose E products (Wolff Cellulosics GmbH & Co. KG, Bomlitz, DE) having a nitrogen content of 11.8% to 12.3% by weight.

Within the aforementioned cellulose-nitric esters of defined nitrogen content, all viscosity levels are suitable in each case. Low-viscosity cellulose-nitric esters with different nitrogen contents are classified into the following groups in accordance with ISO 14446: ≧30A, ≧30M, ≧E. Medium-viscosity cellulose-nitric esters with different nitrogen contents are classified into the following groups in accordance with ISO 14446: 18 E to 29 E, 18 M to 29 M, 18A to 29 A. High-viscosity cellulose-nitric esters with different nitrogen contents, correspondingly, in accordance with ISO 14446 are: ≦17 E, ≦17 M and ≦17 A.

It is also possible to use mixtures of different types of the above-mentioned suitable cellulose-nitric esters.

The nitrocellulose is supplied commercially generally in stabilized form. Examples of typical stabilizers are alcohols or water. The amount of stabilizers is between 5% to 40% by weight. To prepare the dispersions of the invention it is preferred to use nitrocelluloses which have been damped with alcohols or water. One particularly preferred form uses nitrocellulose which has been damped with 10% to 40% by weight of isopropanol (based on the total mass of the as-supplied form). Examples that may be mentioned include “Walsroder® nitrocellulose E 560 isopropanol 30%”, “Walsroder® nitrocellulose A 500 isopropanol 30%” and “Walsroder® nitrocellulose E 560 water 30%”.

The nitrocellulose is preferably added after step B) and before the dispersing in water C). For the addition the nitrocellulose is added in solution in an organic solvent or solvent mixture, more preferably in solution in an aliphatic ketone, and very preferably in solution in acetone.

The polyurethane dispersion relevant to the invention preferably contains 1% to 90%, more preferably 10% to 70% and very preferably 20% to 60% by weight of nitrocellulose.

Finally, in stage E), solvent present in the dispersions is removed by distillation. The pH of the dispersions essential to the invention is typically less than 9.0, preferably less than 8.5, more preferably less than 8.0.

The solids content of the hybrid dispersions essential to the invention is typically 20% to 65%, preferably 25% to 60%, more preferably 30% to 50% and very preferably from 35% to 45% by weight.

The polyurethane-nitrocellulose particles present in the dispersions essential to the invention have an average particle size of 20 to 700 nm, preferably 30 and 400 nm.

Besides the dispersions (I) essential to the invention, which typically serve as primary and/or secondary film-formers in nail varnish formulations, the nail varnishes of the invention may further employ other film-forming polymers (II) that are known to the skilled person (Cosmetics&Toiletries 108, 1988, 70-82), such as toluenesulphonamide-formaldehyde resin, as both primary and secondary resin systems.

Furthermore, the nail varnishes of the invention may also comprise additives (III) such as dyes, pigments, antioxidants, light stabilizers, emulsifiers, defoamers, thickeners, fillers, flow control agents, shelf-life extenders, moisture donors, odourants, free-radical scavengers and thixotropic agents.

In order to adjust rheological properties it is possible as additives to make use, for example, of optionally organically modified clays such as bentonite, montmorillonite, hectorite and smectite.

For the purpose of coloring it is possible as additives to use the color pigments and/or pearlescent pigments and/or dyes that are known per se to the skilled person, such as Sudan Red, DC Red 17, DC Green 6, DC Yellow 11, DC Violet 2, titanium oxide, iron oxide, chromium oxide, cerium oxide, carbon black, mica coated with titanium oxide, with iron oxide, etc.

Depending on the desired pattern of properties and the intended use of the nail varnishes of the invention it is possible for there to be up to 80% by weight of these additives present in the end product, based on total dry matter.

The nail varnishes of the invention can be used for example as a one-coat varnish or else in multicoat systems.

The nail varnish can be applied by the methods known from the prior art, such as by brushing, rolling, pouring, knife coating or spraying, for example.

The present invention further provides for the use of the nail varnishes of the invention for the coating both of fingernails and/or toenails and of fingernail and/or toenail imitations (false nails).

The gloss of the nail varnishes of the invention is 50 to 100 gloss units, preferably 60 to 100 gloss units and more preferably 70 to 100 gloss units measured at an angle of 20° in accordance with DIN 67530 by means of a gloss meter (micro-haze plus, BYK Gardner, Germany).

The curing/drying of the nail varnishes of the invention takes place preferably at room temperature (23° C.), but may also take place at higher or lower temperature. The tack-free state of the varnishes of the invention is achieved at room temperature ≦10 minutes, preferably ≦8 minutes, more preferably ≦5 minutes.

The pendulum hardness of the nail varnishes of the invention measured after 12 h drying at 32° C. is ≧50 s, preferably ≧100 s, more preferably ≧140 s.

### EXAMPLES

Substances used and abbreviations:

Diaminosulphonate: NH₂—CH₂CH₂—NH—CH₂CH₂—SO₃Na (45% in water)

Desmophen® C2200: polycarbonate polyol, OH number 56 mg KOH/g, number-average molecular weight 2000 g/mol (Bayer MaterialScience A G, Leverkusen, D E)

The nitrocellulose products employed respectively were obtained from Wolff Cellulosics GmbH & Co. K G, Walsrode, Germany.

Unless noted to the contrary, all percentages are by weight.

Unless noted to the contrary, all analytical measurements relate to temperatures of 23° C.

The solids contents were determined in accordance with DIN-EN ISO 3251.

Unless expressly mentioned otherwise, NCO contents were determined volumetrically in accordance with DIN-EN ISO 11909.

The average particle sizes of the dispersions were ascertained with the aid of laser correlation spectroscopy measurements (Zetasizer 1000, Malvern Instruments, Malvern, UK).

The pendulum hardnesses were determined on a pendulum-hardness measuring instrument called the “Pendulum hardness tester” from BYK Gardner GmbH, Germany in accordance with DIN EN ISO 1522.

The gloss was determined in accordance with DIN 67530 by means of a “micro-haze plus” gloss meter from BYK Gardner GmbH, Germany, after the respective nail varnish had been drawn down and dried on a black-coloured polyamide plastic substrate.

The statement of the drying time at room temperature after application of one coat to the human fingernail, using a brush, was determined by the point in time after which tackiness of the nail varnish by contact was no longer observable.

### Example 1

199.8 g of a difunctional polyester polyol based on adipic acid and hexanediol and neopentyl glycol (average molecular weight 1700 g/mol, OHN=about 66 mg KOH/g solid) were heated to 65° C. Subsequently at 65° C. 35.3 g of hexamethylene diisocyanate were added over the course of 5 minutes and the mixture was stirred at 100° C. until the theoretical NCO value of 3% was reached. The finished prepolymer was dissolved in 276.0 g of acetone at 50° C. and then a solution of 17.3 g of diaminosulphonate, 2.0 g of ethylenediamine and 66.1 g of water was metered in over the course of 5 minutes. The subsequent stirring time was 15 minutes. Subsequently over the course of 5 minutes a solution of 233.2 g of Walsroder® nitrocellulose E560/IPA 30% and 925.1 g of acetone was added. Dispersion took place by addition of 536.6 g of water over the course of 10 minutes. In a subsequent distillation step the solvents were removed under reduced pressure to give a storage-stable PU dispersion having a solids content of 40.0% and an average particle size of 261 nm.

### Example 2

199.8 g of a difunctional polyester polyol based on adipic acid and hexanediol and neopentyl glycol (average molecular weight 1700 g/mol, OHN=about 66 mg KOH/g solid) were heated to 65° C. Subsequently at 65° C. 35.3 g of hexamethylene diisocyanate were added over the course of 5 minutes and the mixture was stirred at 100° C. until the theoretical NCO value of about 3% was reached. The prepolymer was dissolved in 276.0 g of acetone at 50° C. and then a solution of 19.9 g of diaminosulphonate, 2.0 g of ethylenediamine and 66.1 g of water was metered in over the course of 5 minutes. The subsequent stirring time was 15 minutes. Subsequently over the course of 5 minutes a solution of 234.4 g of Walsroder® nitrocellulose E560/IPA 30% and 925.1 g of acetone was added. Dispersion took place by addition of 538.2 g of water over the course of 10 minutes. In a subsequent distillation step the solvents were removed under reduced pressure to give a storage-stable PU dispersion having a solids content of 41.9% and an average particle size of 154 nm.

### Example 3

184.8 g of Desmophen® C2200, 2.4 g of neopentyl glycol and 12.6 g of dimethylolpropionic acid were heated to 65° C. Subsequently at 65° C. 61.8 g of bis(4,4′-isocyanatocyclohexyl)methane and 10.8 g of isophorone diisocyanate were added over the course of 5 minutes and the mixture was stirred at 100° C. until the theoretical NCO value of 2.76% was reached. The finished prepolymer was dissolved with 9.3 g of triethylamine and 638.3 g of acetone at 50° C. and then a solution of 1.0 g of diethylenetriamine, 0.9 g of ethylenediamine, 2.1 g of hydrazine hydrate and 8.6 g of water was metered in over the course of 10 minutes. The subsequent stirring time was 5 minutes. Subsequently over the course of 5 minutes a solution of 174.4 g of Walsroder® nitrocellulose E330/IPA 30% and 488.3 g of acetone was added. Dispersion took place by addition of 601.9 g of water over the course of 15 minutes. In a subsequent distillation step the solvents were removed under reduced pressure to give a storage-stable PU dispersion having a solids content of 39.0% and an average particle size of 260 nm.

### Example 4

140.0 g of a difunctional polyester polyol based on adipic acid and hexanediol (average molecular weight 840 g/mol, OHN=about 133 mg KOH/g solid), 1.9 g of trimethylolpropane and 14.2 g of 1,6-hexanediol were heated to 65° C. Subsequently at 65° C. 18.1 g of hexamethylene diisocyanate and 99.6 g of bis(4,4′-isocyanatocyclohexyl)methane and also 68.4 g of acetone were added over the course of 5 minutes and the mixture was stirred under reflux conditions until the theoretical NCO value of 2.2% was reached. Finally a further 396.8 g of acetone were added. The dissolved prepolymer was admixed over the course of 5 minutes at 40° C. with a solution of 3.9 g of hydrazine hydrate in 16.8 g of water, followed by stirring for 5 minutes. Thereafter over the course of 10 minutes a solution of 28.4 g of diaminosulphonate and 78.0 g of water were metered in. The subsequent stirring time was 5 minutes. Subsequently over the course of 5 minutes a solution of 176.9 g of WALSRODER NITROCELLULOSE E560/30% IPA and 701.9 g of acetone was added. Dispersion took place by addition of 507.5 g of water over the course of 10 minutes. In a subsequent distillation step the solvents were removed under reduced pressure to give a storage-stable PU dispersion having a solids content of 39.0% and an average particle size of 339 nm.

### Example 5

212.5 g of a difunctional polyesterpolyol based on adipic acid and hexanediol and neopentylglycol (average molecular weight 1700 g/mol, OHN=66 mg KOH/g solids) were heated to 65° C. Subsequently at 65° C. over the course of 5 minutes 37.6 g of hexamethylene diisocyanate were added and the mixture was stirred at 100° C. until the theoretical NCO value of 3.3% was reached. The finished prepolymer was dissolved with 375 g of acetone at 50° C. and subsequently a solution of 20.2 g of diaminosulphonate, 2.2 g of ethylenediamine and 90.0 g of water was metered in over the course of 5 minutes. The subsequent stirring time was 15 minutes. Subsequently over the course of 5 minutes a solution of 268.0 g of Walsroder® Nitrocellulose E 560/water 30% and 893.4 g of acetone was added. Dispersion took place by addition of 458.4 g of water over the course of 10 minutes. In a subsequent distillation step the solvents were removed under reduced pressure to give a storage-stable PU dispersion having a solids content of 41.0% and an average penticule size of 279 nm.

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

### Example 6

The dispersion obtained in Example 1 was drawn down using a film-drawing frame onto a glass substrate, in a wet film thickness of 100 μm, and was dried at 32° C. for 12 h. The performance properties of the inventive nail varnish obtained are reproduced in Table 1.

### Example 7

The dispersion obtained in Example 2 was drawn down using a film-drawing frame onto a glass substrate, in a wet film thickness of 100 cm, and was dried at 32° C. for 12 h. The performance properties of the inventive nail varnish obtained are reproduced in Table 1.

### Example 8

The dispersion obtained in Example 3 was drawn down using a film-drawing frame onto a glass substrate, in a wet film thickness of 100 μm, and was dried at 32° C. for 12 h. The performance properties of the inventive nail varnish obtained are reproduced in Table 1.

### Example 9

Same procedure as in Example 6, with the addition to the resultant dispersion from Example 1 of 10% by weight of butylglycol/water mixture (1:1 weight fractions) as a flow control assistant. The performance properties of the inventive nail varnish obtained are reproduced in Table 1.

TABLE 1
Performance properties of the inventive nail varnishes

| | Pendulum | | |
|---|---|---|---|
| | hardness | | Drying time on |
| Example | [s] | Gloss | fingernail at RT |
| 6 | 67 | 79 | 3 min |
| 7 | 77 | 81 | 3 min |
| 8 | 99 | 72 | 3 min |
| 9 | 182 | 80 | 3 min |

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. An aqueous nail varnish comprising an aqueous dispersion of polyurethane-nitrocellulose particles (I) having an average particle size of 20 to 700 nm.

2. An aqueous nail varnish according to claim 1, wherein the nail varnish contains 5% by weight or less of organic solvents and/or plasticizers based on the overall formulation.

3. An aqueous nail varnish according to claim 1, wherein the polyurethane-nitrocellulose particles have an average particle size of 30 to 400 nm.

4. An aqueous nail varnish according to claim 1, wherein the aqueous dispersions (I) are obtained by
A) preparing one or more isocyanate-functional prepolymer from
A1) one or more organic polyisocyanates
A2) one or more polymeric polyols having number-average molecular weights of 400 to 8000 g/mol and OH functionalities of 1.5 to 6,
A3) one or more hydroxy-functional compounds having molecular weights of 62 to 399 g/mol and
A4) one or more isocyanate-reactive, anionic or potentially anionic and optionally nonionic hydrophilicizing agents,
B) reacting some or all of the free NCO groups, before, during or after the addition of an organic solvent, with
B1) one or more amino-functional compounds having molecular weights of 32 to 399 g/mol and/or
B2) one or more amino-functional, anionic or potentially anionic hydrophilicizing agents, with chain extension,
C) dispersing the prepolymers, before during or after step B), in water, any potentially ionic groups present being converted into the ionic form by full or partial reaction with a neutralizing agent,
D) adding nitrocellulose in the form of a solution in an organic solvent or solvent mixture after step A) but before step C), and
E) distilling the dispersion to remove the organic solvent present.

5. An aqueous nail varnish according to claim 1, further comprising film-forming polymers (II) and/or additives (III).

6. An aqueous nail varnish according to claim 5, wherein the additives are selected from the group consisting of dyes, pigments, antioxidants, light stabilizers, emulsifiers, defoamers, thickeners, fillers, flow control agents, shelf-life extenders, moisture donors, odourants, free-radical scavengers and thixotropic agents.

7. Coatings obtained from aqueous nail varnishes according to claim 1.

8. Coatings according to claim 7, wherein the coating has a gloss of 70 to 100 gloss units measured at an angle of 20° in accordance with DIN 67530.

9. Coatings according to claim 7, wherein the coating has a König pendulum hardness, measured after 12 h drying at 32° C., of ≧50 s.

10. Substrates coated with coatings according to claim 7.

11. A method of coating fingernails or toenails comprising applying aqueous nail varnishes according to claim 1.

12. A method of producing aqueous nail varnishes, comprising
A) preparing one or more isocyanate-functional prepolymer from A1) one or more organic polyisocyanates
A2) one or more polymeric polyols having number-average molecular weights of 400 to 8000 g/mol and OH functionalities of 1.5 to 6,
A3) one or more hydroxy-functional compounds having molecular weights of 62 to 399 g/mol and
A4) one or more isocyanate-reactive, anionic or potentially anionic and optionally nonionic hydrophilicizing agents,
B) reacting some or all of the free NCO groups, before, during or after the addition of an organic solvent, with
B1) one more amino-functional compounds having molecular weights of 32 to 399 g/mol and/or
B2) one or more amino-functional, anionic or potentially anionic hydrophilicizing agents, with chain extension,
C) dispersing the prepolymers, before during or after step B), in water, any potentially ionic groups present being converted into the ionic form by full or partial reaction with a neutralizing agent,
D) adding nitrocellulose in the form of a solution in an organic solvent or solvent mixture after step A) but before step C), and
E) distilling the dispersion to remove the organic solvent present.
